(19) United States
(12) Patent Application Publication
(10) Pub. No.: US 2007/0265692 A1
(43) Pub. Date: Nov. 15, 2007

(54) POROUS SURFACE ELECTRODE FOR CORONARY VENOUS APPLICATIONS

(75) Inventors: Brendan E. Koop, Coon Rapids, MN (US); Shamim M. Malik, Temecula, CA (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/748,614

(22) Filed: May 15, 2007

Related U.S. Application Data
(60) Provisional application No. 60/747,237, filed on May 15, 2006.

(51) Int. Cl. A61N 1/05

(52) U.S. Cl. 607/119

(57) ABSTRACT

The present invention relates to a porous surface electrode including a capacitive layer and a method of making the same. The porous surface electrode of the present invention includes a more thin capacitive coating that preserves the underlying structure of the porous surface. By controlling one or more processing parameters the average thickness and surface morphology of the capacitive coating can be controlled.

### PRIORITY CLAIM

This application claims the benefit under 35 U.S.C. 119(e) of U.S. Provisional Patent Application No. 60/747,237, filed on May 15, 2006, entitled “Porous Surface Electrode for Coronary Venous Applications,” which is incorporated herein by reference in its entirety.

### TECHNICAL FIELD

This invention relates to body implantable medical devices, and more particularly, to implantable electrodes for sensing electrical impulses in body tissue or for delivering electrical stimulation pulses to an organ, for example, for pacing the heart.

### BACKGROUND

Cardiac pacing leads are well known and widely employed for carrying pulse stimulation signals to the heart from a battery operated pacemaker, or other pulse generating means, as well as for monitoring electrical activity of the heart from a location outside of the body. Electrodes are also used to stimulate the heart in an effort to mitigate bradycardia or terminate tachycardia or other arrhythmias. In all of these applications, it is highly desirable to optimize electrical performance characteristics of the electrode/tissue interface. Such characteristics include minimizing the threshold voltage necessary to depolarize adjacent cells, maximizing the electrical pacing impedance to prolong battery life, and minimizing sensing impedance to detect intrinsic signals.

For cardiac pacemaker leads and electrodes, pacemaker implant lifetime may be partially determined by the energy delivered per pulse. Other factors that determine the energy used by the pacemaker include the electrode size, material, surface nature, and shape, the body tissue or electrolyte conductivity, and the distance separating the electrode and the excitable tissue. The pacemaker will have a longer life if the energy delivered per pulse is maintained at a minimum. Alternatively, the saved energy can also be used to provide for more features in the pacemaker.

Pacing (or stimulation) threshold is a measurement of the electrical energy required for a pulse to initiate a cardiac depolarization. The pacing threshold may rise after the development of a fibrous capsule around the electrode tip, which occurs over a period of time after implantation. The thickness of the fibrous capsule is generally dependent upon the mechanical characteristics of the distal end of the lead (i.e. stiff or flexible), the geometry of the electrode tip, the microstructure of the electrode tip, and the biocompatibility of the electrode and other device materials. In addition, the constant beating of the heart can cause the electrode to pound and rub against the surrounding tissue, causing irritation and a subsequent inflammatory response, eventually resulting in a larger fibrotic tissue capsule.

In a pacemaker electrode, minimal tissue reaction is desired around the tip, but firm intimate attachment of the electrode to the tissue is essential to minimize any electrode movement. A porous electrode tip with a tissue entrapping structure allows rapid fibrous tissue growth into a hollow area or cavity in the electrode tip to facilitate and enhance attachment of the electrode to the heart and increase biocompatibility. A reduced electrode dislodgement rate is also expected as a result of such tissue in-growth. A further aspect is selection of the average pore size, which must accommodate economical construction techniques, overall dimensional tolerances, and tissue response constraints. Tissue in-growth may assist in anchoring the electrode in place and increasing the contact surface area between the electrode and the tissue.

One issue with porous electrodes has been that the application of a capacitive coating on porous surface electrodes presents challenges when attempting to ensure uniformity of thickness and coating coverage. A capacitive coating is often placed on an electrode because even normally biocompatible metallic surfaces of many electrodes, when paced, can undergo chemical reactions that may create a toxic byproduct. The coating, made of a material that can inject charge into the tissue without undue accumulation of toxic byproducts, protects the electrode material by preventing the unwanted byproduct formation. Application on a porous surface, however, may result in a shadowing effect, where exposed metallic areas of the electrode remain inside pores, or may result in portions of the pores being closed (filled in) such that tissue in-growth is prevented.

Coronary venous pacing leads are relatively new innovations that have expanded the indications for pacemakers and ICD's. First introduced in the U.S. in 2001 for use in cardiac resynchronization therapy pacemakers (CRT-P), and in 2002 for use in cardiac resynchronization therapy defibrillators (CRT-D), coronary venous pacing leads are used for treating conditions such as heart failure resulting from mechanical dissynchrony of the right and left ventricles. In these applications, coronary venous leads are often called “left ventricular” leads, or “LV” leads, because they are used to pace the left ventricle such that its contractions are synchronized with those of the right ventricle (“resynchronization therapy”).

More recently, implantation in the left side of the heart has been more frequently undertaken to deal with certain specific conditions, such as, for example, heart failure. Patients with heart failure often have dissynchrony between the left and right sides of the heart or may have one side of the heart overgrown as compared to the other side of the heart. In such cases an electrode inserted into the left side may be accomplished by accessing the coronary sinus and implanting the lead in a coronary vein.

Left ventricular lead pacing can present different challenges when compared to right side pacing. First, the electrodes reside in the lumen of the coronary vein and may be further from excitable myocardial tissue than is typical in the right side. Pacing in a coronary vein must be provided through a layer of endothelial cells. Furthermore, pacing must be provided through additional layers of tissue, which may include fat, such that a larger voltage is necessary to create the electric field necessary to depolarize myocardial tissue.

It is therefore important to explore other options for left side electrode placement, such as porous electrodes, for enhancing electrode performance.

### SUMMARY

According to one embodiment, the present invention is a method of forming a capacitive coating on a porous electrode surface for an implantable medical electrical lead. A microporous surface is formed on an electrode having a base material. The microporous surface is exposed to a capacitive material under conditions suitable to sputter deposit the capacitive material onto the microporous surface. One or more sputtering parameters is controlled to form a substantially continuous capacitive coating having an average thickness of less than about 500 nm. According to a further embodiment, the capacitive material includes iridium oxide.

According to another embodiment, the present invention is a medical electrical lead. The medical electrical lead includes a conductive lead body having a proximal end and a distal end and an electrode. The electrode is disposed on the lead body between the proximal end and the distal end. The electrode includes a conductive base material having a microporous surface and a substantially continuous capacitive coating disposed on the microporous surface. The capacitive coating has an average thickness of less than about 500 nm. According to a further embodiment, the thickness of the capacitive coating ranges from about 10 nm to about 300 nm.

According to another embodiment, the present invention is a cardiac rhythm management system. The cardiac rhythm management system includes a pulse generator adapted to deliver a therapy to a patient's heart, a conductive lead body having a proximal end and a distal end, and an electrode. The proximal end of the conductive lead body is operatively coupled to the pulse generator and the distal end is sized and shaped to be disposed within a coronary vein located on the left side of the heart. The electrode is disposed on the lead body between the proximal end and the distal end. The electrode includes a conductive base material having a microporous surface and a substantially continuous capacitive coating disposed on the microporous surface. The capacitive coating has an average thickness of less than about 500 nm.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a lead deployed in a patient's heart according to one embodiment of the present invention.

FIG. 2 is a perspective view of the lead shown in FIG. 1 according to an embodiment of the present invention.

FIG. 3 is a perspective view of an electrode according to an embodiment of the present invention.

FIG. 4 is a side sectional view taken along line A-A of FIG. 3.

FIG. 5 is a side sectional view of an embodiment of FIG. 4 with the addition of a degradable protective coating.

FIG. 6 is a scanning electron microscope image of a surface morphology of a porous electrode including a capacitive coating according to an embodiment of the present invention.

FIG. 7 is a scanning electron microscope image of a surface morphology of a porous electrode including a capacitive coating according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The present invention relates to methods of forming a medical electrical lead having a porous surface electrode and a thin capacitive layer over the electrode.

Leads formed according to embodiments of the present invention may be particularly suitable for placement in a coronary vein in the left side of the heart. Additionally, leads formed according to embodiments of the present invention may have sufficient pacing thresholds for coronary vein placement.

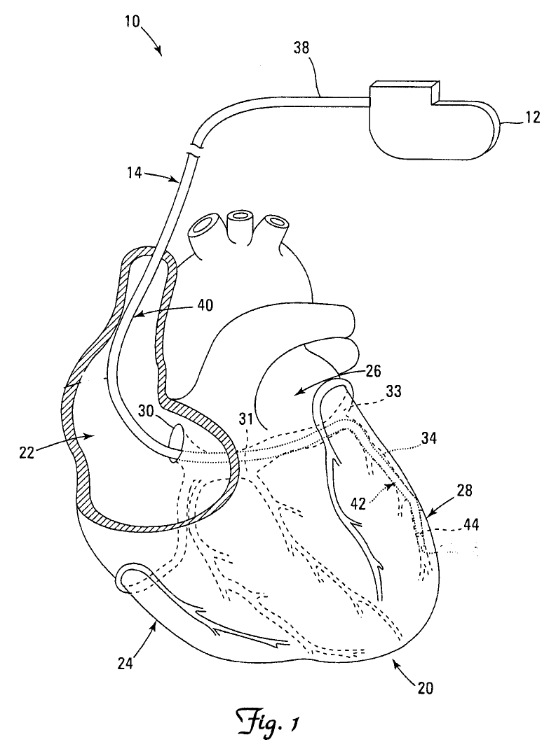

FIG. 1 is a schematic drawing of a cardiac rhythm management system 10 including a pulse generator 12 coupled to a medical electrical lead 14 deployed in a patient's heart 20, which includes a right atrium 22 and a right ventricle 24, a left atrium 26 and a left ventricle 28, a coronary sinus ostium 30 in the right atrium 22, a coronary sinus 31, and various coronary veins including a great cardiac vein 33 and other branch vessels off the coronary sinus 31 including an exemplary branch vessel 34.

According to one embodiment, as shown in FIG. 1, the distal portion 40 is guided through the right atrium 22, the coronary sinus ostium 30 and the coronary sinus 31, and into the branch vessel 34 of the coronary sinus 31. The distal end 46, and thus the electrode 50 is positioned within the branch vessel 34 on the left side of the heart 20. The illustrated position of the lead 14 may be used for delivering a pacing and/or defibrillation stimulus to the left side of the heart 20. Additionally, it will be appreciated that the lead 14 may also be partially deployed in other cardiac vessels such as the great cardiac vein 33 or other branch vessels for providing therapy to the left side or right side of the heart 20.

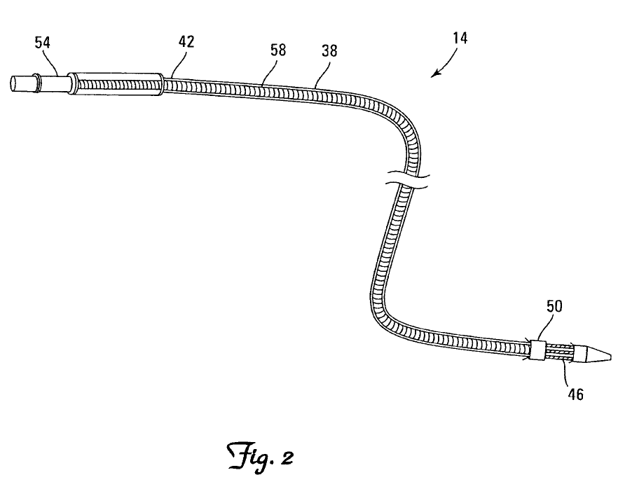

FIG. 2 is a perspective view of the lead 14 shown in FIG. 1. The lead 14 is adapted to deliver electrical pulses to stimulate a heart and/or for receiving electrical pulses to monitor the heart. According to one embodiment of the present invention, the lead 14 is sized and configured to be delivered within a coronary vein located on the left side of the heart 20. The medical electrical lead 14 includes an elongated conductive lead body 38 having opposed proximal and distal ends 42 and 46. The lead body 38 is formed from a bio-compatible insulative material such as silicone rubber, polyurethane, or the like.

A connector 54 is operatively associated with the proximal end 42 of the conductive lead body 38. The connector 54 may be of any standard type, size or configuration. Connector 54 is electrically connected to the electrode 50 by way of a conductor coil 58 that extends through the interior lumen of lead body 38. Conductor coil 58 is generally helical in configuration and includes one or more conductive wires or filaments.

At least one electrode 50 is operatively associated with the distal end 46 of the conductive lead body 38. The electrode 50 can be formed from platinum, stainless steel, MP35N, titanium, a platinum-iridium alloy, or another similar conductive material. In one embodiment, the electrode 50 is disposed proximate to the distal end 46 of the lead 14. Alternatively, the electrode 50 can be located anywhere along the conductive lead body 38 between the proximal end 42 and the distal end 46. According to yet another embodiment of the present invention, the electrode 50 can be a tip electrode. A tip electrode is located at the very distal end 46 of the lead body 38 and is commonly employed in left ventricular leads. Multiple electrodes may also be utilized according to embodiments of the present invention.

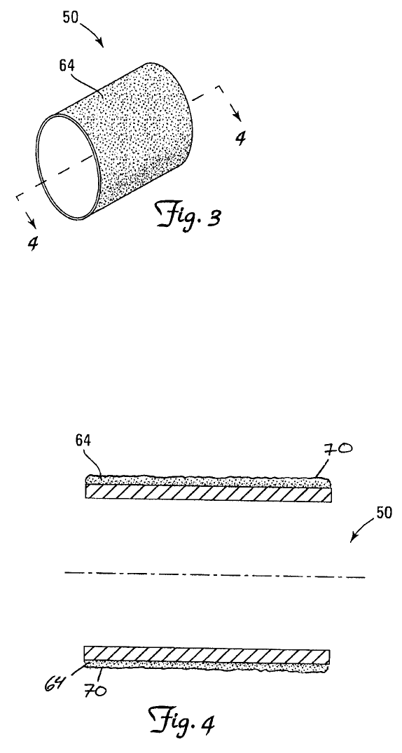

According to exemplary embodiments of the present invention, as shown in FIGS. 3-4, the electrode 50 is covered by a microporous surface 64 and a capacitive coating 70. In one embodiment, the microporous surface 64 is fully disposed over the entire electrode 50. Alternatively, the microporous surface 64 may be partially disposed over the electrode 50. The microporous surface 64 may encourage fibrotic tissue in-growth when implanted. Fibrotic tissue in-growth may assist in anchoring the electrode 50 at a target location within the heart 20 and may also increase the pacing threshold and the sensing capability of the electrode 50 by increasing the surface area in contact with the cardiac tissue. A reduced electrode dislodgement rate may also be accomplished.

According to one embodiment of the present invention, the microporous surface 64 of the electrode 50 is formed by sintering microparticles of a generally conductive material onto the base material of the electrode 50. The microparticles may be made of platinum, platinum iridium, titanium, or another metal compatible with the base electrode material. Sintering is a conventional method for adhering particles to a substrate by heating the material below its melting point until its particles adhere to each other. In one embodiment, the microparticles may be placed on the electrode 50 by packing them around the electrode 50 in a mold. The mold is then heated for a desired period of time until the microparticles adhere to the electrode substrate. In another embodiment, the microparticles may be packed in the mold in the presence of a second material that melts away during the sintering process. The second material may be in a granular form and may include such materials such as polymethylmethacrylate (PMMA). The dissipation of the second material encourages pore formation during the sintering of the microparticles.

Other suitable materials for the particles may include platinum, titanium, and tantalum. In alternative embodiments the particles may be made of any material that is compatible with the base material of the electrode 50. In one embodiment, the particles are formed or supplied as microspheres, however, other shapes may also be suitable. Mircospheres made from a wide variety of materials are commercially available from a variety of sources.

In addition to sintering, other processes for forming the microporous surface 64 can include plasma spray coating, liquid metal coating, electrode burning, laser scribing, acid etching, mechanical abrasion, particle blasting, thermal spray coating, plasma etching, diamond coating, and powder metallurgy such as a casting or forming processes.

In a further embodiment of the present invention, the average pore size of the porous surface 64 may be optimized for placement in the coronary veins, which generally require smaller lead sizes. The average pore size may be further optimized based on the degree of tissue in-growth desired. An optimized average pore size for the coronary venous electrode may be between about 1 and 200 microns. In further embodiments, the average pore size may be between about 50 and about 100 microns.

In yet further embodiments of the present invention, various mechanical features may be incorporated into the porous surface 64 formed on the electrode 50. Such features may include ridges, holes, abrasion areas (such as formed via grit blasting or chemical etching), voids, steps, etc. Mechanical features formed in the porous surface 64 of the electrode 50 may contribute to electrode 50 retention and fixation stability. Such electrodes 50 may include a macro primary mechanical feature formed from the sintered spheres. A structure may be formed from a process such as highly energetic ion bombardment, for example, with argon in a plasma assisted process, such that surface features and porosity on the order of 100 nm to 10 μm are formed via bubbling or reformation of the metallic surface. In still further embodiments, tertiary structural features may be included through deposition of the capacitive coating 70 or a degradable protective coating 80. In addition, the electrode 50 as a whole may have a fractal morphology.

As shown in FIG. 4, a capacitive coating 70 is deposited on the microporous surface 64. The capacitive coating 70 may provide improved electrochemical characteristics at the site of electrode implantation by altering the electrochemistry at the electrode/electrolyte interface. Capacitive coatings are corrosion resistant and electrochemically stable, and, may allow stimulation by charging and discharging of the Helmholtz double layer that is formed across the coating. The amount of charge injected in this manner is proportional to the electrochemically active surface area of the electrode. Further morphological alteration of the coated surface can raise its charge injection capacity. The choice of coating material is also important, as some materials can also inject a portion of the total charge via reversible faradaic processes that result in minimal amounts of new substances being formed.

According to one embodiment of the present invention, the capacitive coating 70 includes a biocompatible metal or metallic compound. Exemplary compounds include platinum, tantalum, platinum iridium, platinum oxide, titanium oxynitride, titanium oxide, tantalum oxide, tantalum nitride, titanium carbide, iridium oxide, and combinations thereof. Other biocompatible metallic compounds known to those in the art also can be used. According to alternative embodiments of the present invention, the capacitive coating 70 can be a capacitive polymer derived from, for example, poly(pyrrole), poly(naphthalene), poly(thiophene), PEDOT, Nafion, poly(ethylene) oxide or other suitable polymers.

There are many processes that can be used to grow or deposit capacitive coatings, such as electrodeposition, electroactivation, thermal deposition, and sputtering. Additional processes that can be used include plasma polymerization, chemical vapor deposition (CVD), and plasma enhanced chemical vapor deposition (PECVD). All of these processes involve a myriad of inputs that each affect the eventual composition and morphology of the coating. The coating composition and its morphology, in turn, establish the electrically active surface area of the electrode (as opposed to the geometric surface area, based on the physical dimensions of the electrode).

The surface morphology of the capacitive coating may affect cellular attachment and cellular proliferation which may provide fixation of the electrode in vivo. For example, see Webster et al., “Osteoblast adhesion on nanophase ceramics,” Biomaterials, vol. 20, pp. 1221-1227, 1999; Chehroudi et al., “Effects of a grooved titanium-coated implant surface on epithelial cell behaviour in vitro and in vivo,” J. Biomed. Mat. Res., vol. 23, pp. 1067-1085, 1989; and Campbell et al., “Microtopography and soft tissue response,” J. Investigative Surg., vol. 2, pp. 51-74, 1989.

According to exemplary embodiments of the present invention, various sputtering deposition techniques are used to deposit the capacitive coating 70 on the microporous surface 64. Sputtering is a process by which thin films of uniform thicknesses may be deposited onto a substrate by accelerating ions, typically via glow discharge plasma, toward a “target” material such that atoms from the target are ejected and deposited on the substrate surface. If the plasma gas is chosen such that the sputtered atoms react with the ions in the plasma to form a new compound, which is then deposited on the surface of the substrate, the process is called “reactive” sputtering. In the case of a pacing electrode, the electrode serves as the substrate.

A variety of commercially available sputtering system designs may be used to accomplish the desired coating deposition. An example of one system is a cylindrical magnetron sputter deposition system. In such a system, a “target” is situated against the wall of a cylindrical vacuum chamber. The target serves as the source of metallic ions in the chamber once a low pressure glow discharge plasma is formed, which bombards the target surface with energetic ions that cause ejection of metallic target ions. The target may be made up of any number of metals, such as iridium, titanium, tantalum, etc. The sputtered target ions travel in random directions through the vacuum chamber, and may combine with a reactive gas that is present in the plasma to form a compound. The reactive gas is most usually oxygen or nitrogen, which is present typically with a portion of argon, which serves as an inert gas for sputtering.

The compound is often formed on the substrate, which sits at the center of the chamber, and may be biased with a voltage to attract sputtered ions and gas ions to form a compound coating on the surface. According to one embodiment, the present invention utilizes pulsed voltages to assist in uniformly depositing the capacitive coating 70. According to one embodiment of the present invention, the pulsed voltage applied to the substrate ranges from about −100 V to about −300 V. In further embodiments of the present invention, the pulsed voltages are induced in a square wave pattern with frequencies less than about 100 kHz.

Various processing parameters present in the sputtering system control the thickness and/or surface morphology of the compound coating on the substrate, as well as the composition of constituent materials (i.e. target metal and reactive gas atoms). Such parameters include the input power to the magnetron, the ratio of reactive gas to inert gas in the chamber, processing time, and chamber pressure.

According to one exemplary embodiment of the present invention the magnetron input power can range from about 100 W to about 300 W. In one particular embodiment, the magnetron power ranges from about 175 W to about 225 W.

The ratio of reactive gas (e.g. oxygen or nitrogen) to inert gas (e.g. argon) can range from about 15% (oxygen) to about 45% (oxygen). According to another embodiment of the present invention, the ratio of reactive gas to inert gas ranges from about 20% (oxygen) to about 30% (oxygen). In yet another exemplary embodiment of the present invention, the ratio of reactive gas to inert gas ranges from about 35% (oxygen) to about 45% (oxygen).

The processing time can range from about 30 seconds to about 4 minutes. According to another embodiment of the present invention, the processing time ranges from about 1 minute to about 2 minutes. According to yet another embodiment of the present invention, the processing time ranges from about 2.5 minutes to about 3.5 minutes.

The total chamber pressure can range from about 0.1 mTorr to about 15 mTorr. According to another embodiment of the present invention, the total chamber pressure ranges from about 3 mTorr to about 5 mTorr. According to yet another embodiment of the present invention, the total chamber pressure ranges from about 9 mTorr to about 11 mTorr.

In one embodiment, the capacitive coating 70 is formed under processing parameters that produce an average thickness at the porous electrode surface 64 that is much less than the average pore size or characteristic feature size of the porous surface, such that the underlying surface structure of the microporous surface 64 is substantially preserved. According to one embodiment of the present invention, an average thickness of the capacitive coating is less than about 500 nm. According to another exemplary embodiment, the average thickness of the capacitive coating ranges from about 10 nm to about 300 nm. According to a further embodiment of the present invention, the thickness of the capacitive coating ranges from about 150 nm to 300 nm. According to yet a further embodiment of the present invention, the thickness of the capacitive coating ranges from about 20 nm to about 60 nm. The resultant capacitive coating 70 is more complete and uniform across the porous surface 64 and provides better protection for the electrode 50 from the surrounding cardiac tissue.

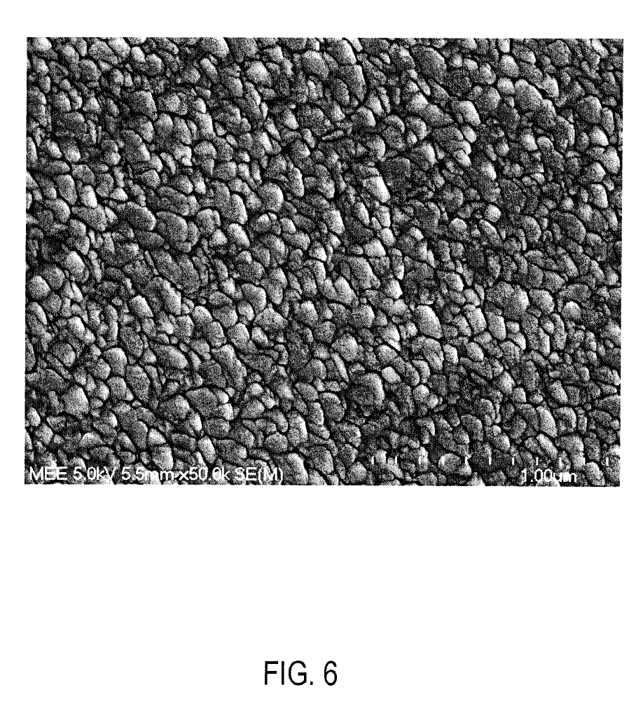

According to one particular embodiment of the present invention, a capacitive coating 70 having a thickness ranging from about 25 nm to about 50 nm and a surface morphology that has an “orange peel” like appearance, as shown in FIG. 6, can be achieved using the following processing conditions: a total chamber pressure ranging from about 3 mTorr to about 5 mTorr; a oxygen to argon gas ratio of about 20% to about 30%; and a processing time of about 1 minute to about 2 minutes.

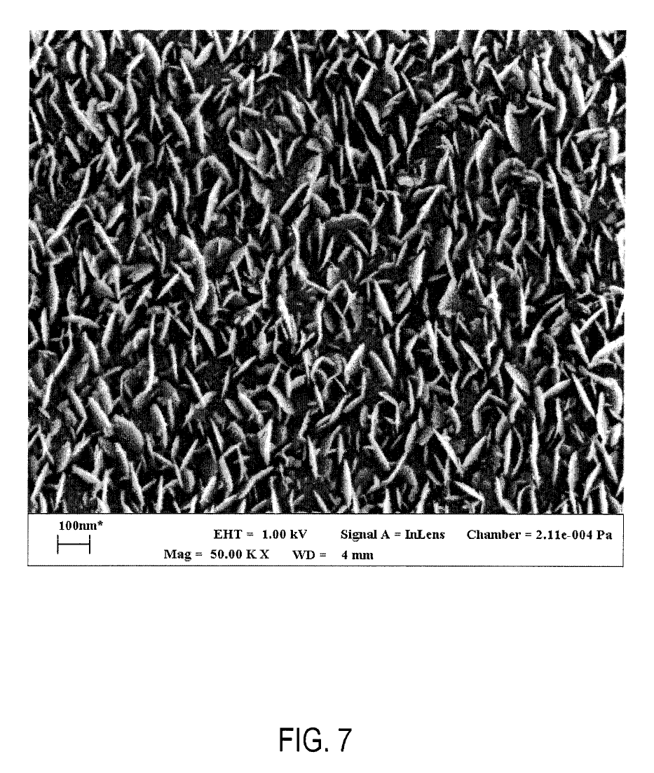

According to another particular embodiment of the present invention, a capacitive coating 70 having a thickness ranging from about 285 nm to about 295 nm and a surface morphology that has a “rice grain” like appearance, as shown in FIG. 7, can be achieved using the following processing conditions: a total chamber pressure ranging from about 9 mTorr to about 11 mTorr; a oxygen to argon gas ratio of about 35% to about 45%; and a processing time of about 2.5 minutes to about 3.5 minutes.

Post processing procedures, such as annealing or electrochemical activation, may also be performed to further optimize the capacitive coating 70 surface morphology.

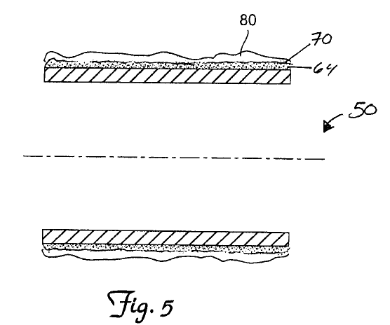

In still further embodiments, as illustrated in FIG. 5, the electrode 50 may include a degradable protective coating 80 over the capacitive coating 70. The degradable protective coating 80 degrades over a pre-determined period of time to protect the electrode 50 from the formation of thrombi or other biological responses during the implantation procedure. In various embodiments the degradable protective coating 80 may be completely degraded before or after the electrode 50 is in the final desired position.

According to one such embodiment of the present invention, the degradable protective coating 80 includes a hydrogel. The hydrogel coating may be constructed of a biocompatible material such as poly(ethylene glycol) (PEG). Hydrogel protective coatings may aid or mediate tissue in-growth before it is completely degraded. In addition, the degradable, hydrogel protective coating may serve to protect the porous electrode from blood coagulation during placement of the lead such that a preliminary thrombus does not form until after the electrode is in the desired position. Many hydrogels, such as those formed from PEG, may also display lubricious characteristics and may therefore further aid in the insertion of the porous electrode into the coronary vein.

The PEG polymer may be in a variety of forms such as a copolymer, a cross-linked network of polymers, a graft polymer, or a polymer blend. PEG comes in a variety of high or low molecular weights which can be selected depending on the desired degradation properties. PEG can be deposited on the porous surface electrode 50 by dip coating the porous surface electrode. In further embodiments of the present invention, a solution containing the polyethylene glycol may be applied to the electrode 50 via a syringe.

In still further embodiments of the present invention, the surface of the electrode 50 may be protected via other degradable materials such as mannitol, or other degradable polymers, such as polylactic-co-glycolic acid (PLGA) or any polymer. The polymer is selected such that its surface or bulk degradation properties can be adjusted so as to dissolve over time to protect the porous surface 64 from thrombus formation or other undesirable biological processes until the electrode is in its final implant position.

The drug eluting properties of certain materials that can be used as the degradable protective coating 80 may also provide a site specific vehicle for delivery of drugs and other biologically active agents. Such agents may help to further reduce blood clotting and thrombi formation. According to one exemplary embodiment, one drug that may be desirable to load directly into or on the degradable protective coating 80, or the porous surface 64, includes dexamethazone acetate. Dexamethazone acetate is a anti-inflammatory agent and may contribute to the reduction of fibrous capsule growth and decrease the pacing threshold.

In further embodiments of the present invention, degradable protective coating materials 80 may be formed as a copolymer or a blend with other polymers to achieve desired degradation or drug eluting characteristics. Other drugs may be loaded into the pores, doped into or onto the degradable polymer and/or applied to the capacitive polymer coating. Further drugs may include, for example, clobetesol, everoliums, sirolimus, or dexamethazone phosphate.

### EXAMPLES

### Example 1

A cylindrical DC magnetron sputtering system (ion Tech, Inc., Fort Collins, Colo.) was used to reactively sputter an iridium oxide capacitive coating onto a stainless steel test strip. The sputtering target was iridium (99.9% purity). Iridium targets are commercially available from a variety of sources. The sputtering parameters used to form the thin film are summarized below in Table 1.

TABLE 1

| | |
|---|---|
| Base Pressure: | 5.00 × 10−6 torr |
| Pre Cleaning | Argon Flow = 35.00 sccm |
| | Chamber Pressure = 37 mTorr |
| | Glow Discharge PS by Power = 25 W |
| | Cleaning Time = 5 minutes |
| Magnetron | Magnetron Power: 200 W |
| Cleaning | Shield closed |
| | Time = 4 minutes |
| IROX Deposition | Shield open |
| | Chamber Pressure Control = 4 mTorr |
| | Oxygen % = 25% (percentage of total gas flow, rest |
| | is Argon) |
| | Process Time = 105 seconds |

FIG. 6 is a scanning electron microscope image of the resultant iridium oxide capacitive coating. As shown in FIG. 6, the surface morphology of the capacitive coating has an “orange peel” like appearance. Cross sections of the coated substrates were created using Focused Ion Beam (FIB). The thickness of the capacitive coating was then evaluated using a scanning electron microscope. The average thickness of the deposited capacitive coating was about 25 nm to about 50 nm.

### Example 2

A cylindrical DC magnetron sputtering system (ion Tech, Inc., Fort Collins, Colo.) was used to reactively sputter an iridium oxide capacitive coating onto a stainless steel test strip. The sputtering target was iridium (99.9% purity). Iridium targets are commercially available from a variety of sources. The sputtering parameters used to form the thin film are summarized below in Table 2.

TABLE 2

| | |
|---|---|
| Base Pressure: | 5.00 × 10−6 torr |
| Pre Cleaning | Argon Flow = 35.00 sccm |
| | Chamber Pressure = 37 mTorr |
| | Glow Discharge PS by Power = 25 W |
| | Cleaning Time = 5 minutes |
| Magnetron Cleaning | Magnetron Power: 200 W |
| | Shield closed |
| | Time = 4 minutes |
| IROX Deposition | Shield open |
| | Chamber Pressure Control = 10 mTorr |
| | Argon Flow = 30.00 sccm |
| | Oxygen Flow = 20.00 sccm |
| | Process Time = 3 minutes |

FIG. 7 is a scanning electron microscope image of the resultant iridium oxide capacitive coating. As shown in FIG. 7, the surface morphology of the capacitive coating looks similar to tiny grains of rice. Cross sections of the coated substrates were created using Focused Ion Beam (FIB). The thickness of the capacitive coating was then evaluated using a scanning electron microscope. The average thickness of the deposited capacitive coating was 293 nm.

By controlling the sputtering parameters, the thickness and/or surface morphology of the capacitive coating 70 were controlled. Altering the ratio of argon to oxygen in the total flow rate appears to most significantly impact the surface morphology of the capacitive layer. The chamber pressure also affects the surface morphology. Increasing the processing time of the sample increases the thickness of the capacitive layer.

The scope of the invention is not meant to be limited in application only to leads for implantation in coronary veins. Application of the disclosed embodiments may also be made to right sided bradycardia or tachycardia leads, or epicardial leads. For coronary venous applications, the disclosed embodiment may also be utilized on a non-electrode metallic portion of the lead body strictly for tissue integration and lead stabilization.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of forming a capacitive coating on a porous electrode surface for an implantable medical electrical lead, the method comprising:
providing an electrode having a conductive base material;
forming a microporous surface on the conductive base material of the electrode;
exposing the microporous surface to a capacitive material under conditions suitable to sputter deposit the capacitive material onto the microporous surface; and
controlling one or more sputtering parameters to form a substantially continuous capacitive coating having an average thickness of less than about 500 nm.

2. The method of claim 1, wherein depositing the controlling step comprises controlling a processing time parameter, a gas flow parameter, a chamber parameter, or a combination thereof.

3. The method of claim 1, wherein the controlling step further comprises controlling a ratio of oxygen to argon in a total gas flow.

4. The method of claim 3, wherein the ratio of oxygen to argon in ranges from about 15% to about 45% oxygen in the total gas flow.

5. The method of claim 1, wherein the controlling step comprises controlling a processing time.

6. The method of claim 5, wherein the processing time ranges from about 30 seconds to about 4 minutes.

7. The method of claim 1, wherein the controlling step further comprises controlling a chamber pressure.

8. The method of claim 7, wherein the chamber pressure ranges from about 0.1 mTorr to about 15 mTorr.

9. The method of claim 1, wherein the exposing step further comprises inducing a pulsed voltage in a square wave form pattern at a frequency of less than about 100 kHz.

10. The method of claim 1, wherein the capacitive material includes iridium oxide.

11. The method of claim 1, further comprising the step of electrochemically activating the capacitive coating.

12. The method of claim 1, further comprising the step of depositing a degradable, protective coating over the capacitive coating.

13. The method of claim 1, further comprising forming the microporous surface to provide an average pore size ranging from about 1 μm to about 200 μm.

14. The method of claim 1, wherein the controlling step forms a capacitive coating having an average ranging from about 10 nm to 300 nm.

15. A medical electrical lead comprising: a conductive lead body having a proximal end and a distal end; and
an electrode disposed on the lead body between the proximal end and the distal end, the electrode including a conductive base material having a microporous surface and a substantially continuous capacitive coating disposed on the microporous surface, wherein the capacitive coating has an average thickness of less than about 500 nm.

16. The medical electrical lead of claim 15, wherein an average pore size of the microporous surface ranges from about 1 μm to about 200 μm.

17. The medical electrical lead of claim 15, wherein the thickness of the capacitive coating ranges from about 10 nm to 300 nm.

18. The medical electrical lead of claim 15, wherein the capacitive coating comprises a material selected from the group consisting of platinum, titanium, tantalum, platinum iridium, platinum oxide, titanium carbide, titanium oxynitride, titanium oxide, tantalum oxide, tantalum nitride, and iridium oxide.

19. The medical electrical lead of claim 15, wherein the capacitive coating comprises iridium oxide.

20. A cardiac rhythm management system comprising:
a pulse generator adapted to deliver a therapy to a patient's heart;
an conductive lead body including a proximal end and a distal end, the proximal end operatively coupled to the pulse generator and the distal end sized and shaped to be disposed within a coronary vein located on the left side of the patient's heart; and
an electrode disposed on the lead body between the proximal end and the distal end, the electrode including a conductive base material having a microporous surface and a substantially continuous capacitive coating disposed on the microporous surface, wherein the capacitive coating has an average thickness of less than about 500 nm.
